(19) United States
(12) Patent Application Publication
(10) Pub. No.: US 2017/0224512 A1
(43) Pub. Date: Aug. 10, 2017

(54) SYSTEMS AND METHODS TO AFFECT THE FLOW OF CHYLOMICRON LIPIDS

(71) Applicant: Boston Scientific Scimed, Inc., Maple Grove, MN (US)

(72) Inventor: John A. Hingston, Framingham, MA (US)

(21) Appl. No.: 15/425,900

(22) Filed: Feb. 6, 2017

Related U.S. Application Data
(60) Provisional application No. 62/291,888, filed on Feb. 5, 2016.

(51) Int. Cl. A61F 5/00

(52) U.S. Cl. CPC A61F 5/0073

(57) ABSTRACT

The present disclosure relates to the field of weight loss. Specifically, the present disclosure relates to systems and methods for reducing daily caloric loads by limiting the delivery of long chain fatty acids into the bloodstream. More specifically, the present disclosure relates to systems and methods for reducing or restricting the flow of chylomicron lipids from the cysterna chyli through the thoracic duct into the bloodstream.

### CROSS-REFERENCE TO RELATED APPLICATIONS

This application claims the benefit of priority to U.S. Provisional Application Ser. No. 62/291,888, filed Feb. 5, 2016, the disclosure of which is herein incorporated by reference in its entirety.

### FIELD

The present disclosure relates to the field of weight loss. Specifically, the present disclosure relates to systems and methods for reducing caloric loads by limiting the delivery of long chain fatty acids into the bloodstream. In particular, the present disclosure relates to systems and methods for reducing or restricting the flow of chylomicron lipids from the cysterna chyli through the thoracic duct into the bloodstream.

### BACKGROUND

An excess of ingested calories may lead to obesity which is associated with a variety of chronic diseases and related physiologic dysfunctions. This is especially true in Western and developing countries. Depending on an individual's particular diet, calories in the form of long chain fatty acids (LCFA) and cholesterol may account for a significant portion of the daily caloric load. These LCFAs and cholesterol molecules are too large and hydrophobic to enter the blood stream directly after absorption in the small intestine. Rather, the LCFAs are reprocessed as chylomicron lipids in the cells that line the gut mucosa and then delivered from the basal surface of those cells into the lacteal ducts of the lymphatic system that lie in close proximity within the lamina propria layer. The lacteal ducts converge into larger channels, then to the lymph nodes and eventually funnel into a variable-sized structure called the cysterna chyli located at the bottom of the thoracic duct. The chylomicron lipids are pumped from the cysterna chyli through the thoracic duct to enter the venous bloodstream at the junction of the left subclavian and jugular veins.

It is thought that slowing the flow of such lipids into the bloodstream might beneficially affect weight loss or gain. There is therefore a need for systems and methods, which interrupt the flow of chylomicron lipids through the thoracic duct into the circulatory system to reduce their availability for cellular metabolism.

### SUMMARY

The present disclosure, in its various aspects, meets an ongoing need in the field of weight loss for reducing daily caloric loads by limiting the delivery of long chain fatty acids into the bloodstream.

In one aspect, the present disclosure relates to a medical device for affecting fluid flow through a body lumen, comprising a stricture element configured to be placed in contact with a portion of the thoracic duct or cysterna chyli. The stricture element may be configured to be placed within the thoracic duct or cysterna chyli. The stricture element may include a proximal end portion; a distal end portion; a medial portion; and a lumen extending therebetween. The stricture element may be configured to move from a first collapsed configuration to a second expanded configuration within the thoracic duct or cysterna chyli. A diameter of the lumen of the medial portion may be less than a diameter of the lumen of the proximal and distal end portions when the stricture element is in the second expanded configuration. The stricture element may be configured to be placed around the thoracic duct or cysterna chyli. The stricture element may include a band configured to resist outward pressure on the thoracic duct or cysterna chyli to create a stricture therein. The band may further include a lumen fluidly connected to a fluid source, such that flowing a fluid from the fluid source into the lumen of the band increases a constrictive pressure on the thoracic duct or cysterna chyli, and flowing a fluid from the lumen of the band into the fluid source decreases a constrictive pressure on the thoracic duct or cysterna chyli. The stricture element may be configured to sealingly contact the thoracic duct or cysterna chyli to reduce the flow of chylomicron lipids through the thoracic duct or cysterna chyli. The stricture element element may be configured to sealingly contact the thoracic duct or cysterna chyli to reduce the flow of chylomicron lipids through the thoracic duct or cysterna chyli by at least 50 percent; by at least 60 percent; by at least 70 percent; by at least 80 percent; by at least 90 percent. Alternatively, the stricture element may be configured to block the flow of chylomicron fluids (i.e., reduce flow by 100 percent) through the thoracic duct or cysterna chyli. The stricture element may be removable. The stricture element may be formed from a biodegradable or bioabsorbable material.

In another aspect, the present disclosure relates to a method for affecting fluid flow through the thoracic duct of a patient, comprising contacting a stricture element with a portion of the thoracic duct to create a flow-affecting stricture therein, wherein the stricture element is placed within the thoracic duct. The stricture element may reduce, or block, the flow of chylomicron lipids through the thoracic duct. The stricture element may be advanced to the thoracic duct through a vessel of the patient; including, for example, the left subclavian vein. The stricture element may include: a proximal end portion; a distal end portion; a medial portion; and a lumen extending therebetween. The stricture element may be configured to move from a first collapsed configuration to a second expanded configuration. A diameter of the lumen of the medial portion may be less than a diameter of the lumen of the proximal and distal end portions when the stricture element is in the second expanded configuration.

In another aspect, the present disclosure relates to a method for affecting fluid flow through the thoracic duct of a patient, comprising: contacting a stricture element with a portion of the thoracic duct to create a flow-affecting stricture therein, wherein the stricture element is placed around the thoracic duct. The stricture element may reduce, or block, the flow of chylomicron lipids through the thoracic duct. The stricture element may be placed around the thoracic duct laparoscopically. The stricture element may include a band configured to resist outward pressure on the thoracic duct creating a stricture. The band may further include a lumen fluidly connected to a fluid source, such that flowing a fluid from the fluid source into the lumen of the band increases a resistance to outward pressure or increases a constrictive pressure on the thoracic duct, and flowing a fluid from the lumen of the band into the fluid source decreases a resistance to outward pressure or decreases a constrictive pressure on the thoracic duct.

In another aspect, the present disclosure relates to a method for affecting fluid flow through a cysterna chyli of a patient, comprising contacting a stricture element with a portion of the cysterna chyli to create a flow-affecting stricture therein, wherein the stricture element is placed within or around the cysterna chyli. The stricture element may reduce, or completely block, the flow of chylomicron lipids through the cysterna chyli. For example, the stricture element may reduce the flow of chylomicron lipids through the cysterna chyli by at least 50 percent (e.g., by at least 60 percent; by at least 70 percent; by at least 80 percent; by at least 90 percent).

### BRIEF DESCRIPTION OF THE DRAWINGS

Non-limiting embodiments of the present disclosure are described by way of example with reference to the accompanying figures, which are schematic and not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 is a schematic illustration of an abdominal cavity including the small intestine, lacteal ducts, lymph nodes, cysterna chyli and thoracic duct.

FIGS. 2A-2B provide side schematic views of a stricture element disposed within a portion of the thoracic duct, according to an embodiment of the present disclosure.

FIGS. 3A-3C provide side schematic views of a stricture element disposed circumferentially around an outer portion of the thoracic duct, according to another embodiment of the present disclosure.

It is noted that the drawings are intended to depict only typical or exemplary embodiments of the disclosure. It is further noted that the drawings may not be necessarily to scale. Accordingly, the drawings should not be considered as limiting the scope of the disclosure. The disclosure will now be described in greater detail with reference to the accompanying drawings.

### DETAILED DESCRIPTION

The present disclosure is not limited to the particular embodiments described. The terminology used herein is for the purpose of describing particular embodiments only, and is not intended to be limiting beyond the scope of the appended claims. Unless defined otherwise, all technical terms used herein have the same meaning as commonly understood by one of ordinary skill in the art to which the disclosure belongs. Finally, although embodiments of the present disclosure are described with specific reference to reducing or restricting the flow of chylomicron lipids through the thoracic duct, it should be appreciated that such the systems and methods disclosed herein may be used to reduce or restrict the flow of biological components through a variety of bodily organs and/or lumens, including for example, the stomach, esophagus, large intestine, small intestine, urinary system, respiratory system, reproductive system, lymphatic system and/or circulatory system.

As used herein, the singular forms “a,” “an” and “the” are intended to include the plural forms as well, unless the context clearly indicates otherwise. It will be further understood that the terms “comprises” and/or “comprising,” or “includes” and/or “including” when used in this specification, specify the presence of stated features, regions, steps, elements and/or components, but do not preclude the presence or addition of one or more other features, regions, integers, steps, operations, elements, components, and/or groups thereof.

As used herein, the term “distal” refers to the end farthest away from a medical professional when introducing a device into a patient, while the term “proximal” refers to the end closest to the medical professional when introducing a device into a patient.

As used herein, the term “expanded” refers to an increase in size, diameter or profile as compared to the size, diameter or profile in an “unexpanded” or “collapsed” configuration.

The present disclosure provides systems and methods for reducing or restricting the flow of long chain fatty acids, lipids and cholesterol from the cysterna chyli through the thoracic duct and into the venous bloodstream. While differing in their specific mechanism of action, each embodiment disclosed herein generally involves placing a stricture element in contact with an internal or external portion of the thoracic duct to create a flow-reducing stricture therein. It should be appreciated, however, that the present disclosure may also include embodiments in which the stricture element is placed in complete or partial contact with an internal or external portion of the cysterna chyli to create a flow-reducing stricture therein.

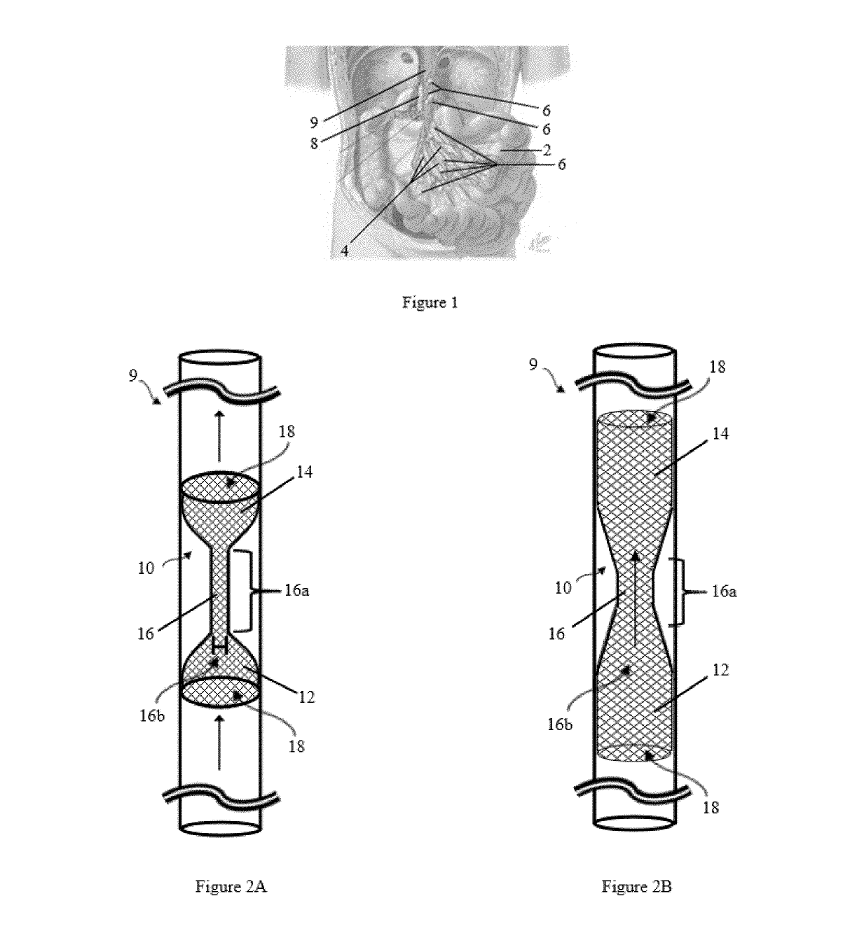

FIG. 1 is a schematic illustration of the abdominal cavity. LCFAs and cholesterol molecules that are too large and hydrophobic to directly enter the blood stream after absorption in the small intestine 2 are reprocessed as chylomicron lipids in the mucosal cells that line the small intestine. The chylomicron lipids are then delivered from the basal surface of the mucosal cells into the lacteal ducts 4 of the lymphatic system where they converge into larger channels, then to the lymph nodes 6 and eventually funnel into the cysterna chyli 8 located at the bottom of the thoracic duct 9. The chylomicron lipids are pumped from the cysterna chyli 8 up the thoracic duct 9 to enter the venous bloodstream at the junction of the left subclavian and jugular veins (not shown).

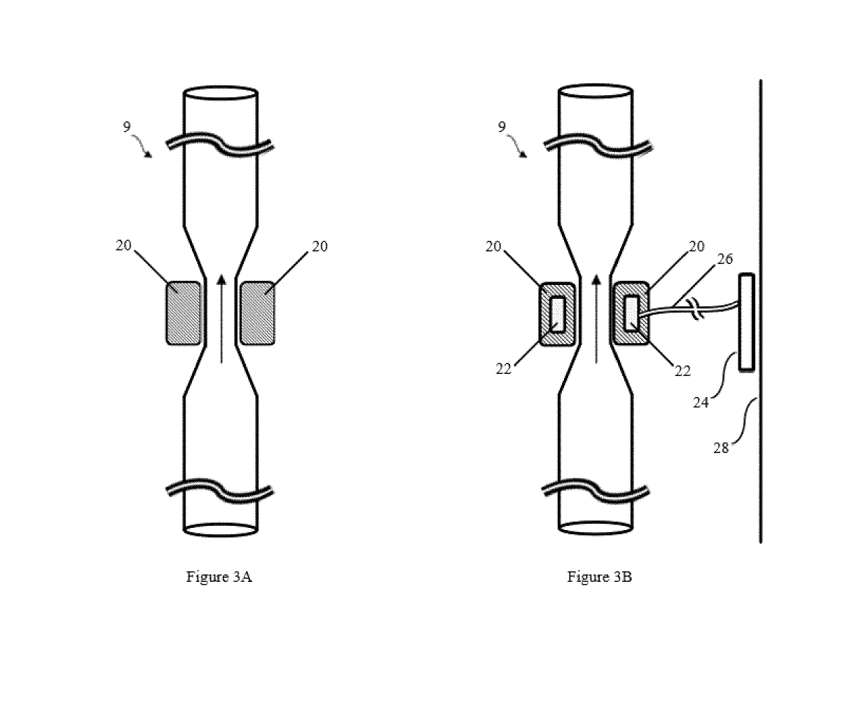

FIG. 2A generally depicts a stricture element 10 disposed within the thoracic duct 9 distal to the intersection with the subclavian vein (not shown). In one embodiment, the stricture element 10 may include a stent-like structure comprising a proximal end portion 12, distal end portion 14, medial portion 16 and a lumen 18 extending therebetween. The stent-like structure 10 may be configured to move from a first (i.e., collapsed/delivery) configuration (not shown) to a second (i.e., expanded/deployed) configuration. When in the second configuration the proximal and distal end portions 12, 14 of the stent-like structure 10 may expand to exert a radial outward force against a portion of the inner wall of the thoracic duct 9, thereby immobilizing the stent-like structure within the thoracic duct. In addition, the proximal and distal end portions 12, 14 form a seal against the thoracic duct such that the lipids and cholesteral flow through the lumen 18 of the stent-like structure 10. The lack of peristaltic movement within the thoracic duct minimizes the likelihood of the stent-like structure migrating within the thoracic duct after deployment.

FIG. 2B generally depicts another embodiment of a stricture element 10 disposed within the thoracic duct 9. As compared to the stricture element of FIG. 2A, the proximal and distal end portions 12, 14 are relatively longer than the medial portion 16, thereby providing a larger surface area to engage and seal against the inner wall of the thoracic duct 9. Similarly, the relatively shorter length of the medial portion 16 prevents or minimize the likelihood of blockages forming within the stricture element 10.

In either of these embodiments, the medial portion 16 of the stent-like structure 10 includes an outer diameter that is less than the outer diameter of the proximal and distal end portions 12, 14 when in the expanded configuration. The reduced diameter 16b of the lumen 18 within the medial portion 16 creates a stricture that reduces the flow of lymph and chylomicron lipids through the thoracic duct. Alternatively, or in addition, the outer diameter of the stricture element 10 may be substantially the same along its length, and include an internal valve, baffle or similar diameter reducing structure that reduces the flow of lymph and chylomicron lipids therethough. The thoracic duct of a typical adult is approximately 38-45 cm in length and has an outer diameter of approximately 6.0 mm or less (e.g., 5.5 mm or less; 5.0 mm or less; 4.5 mm or less; 4.0 mm or less; 3.5 mm or less) and an inner diameter of approximately 3.0 mm or less (e.g., 2.5 mm or less; 2.0 mm or less; 1.5 mm or less; 1.0 mm or less; 0.5 mm or less). In one embodiment, the stent-like structure 10 may reduce the flow of lymph and chylomicron lipids through the thoracic duct by at least 10 percent (e.g., by at least 20 percent; by at least 30 percent; by at least 40; by at least 50 percent; by at least 60 percent; by at least 70 percent; by at least 80 percent; by at least 90 percent). In another embodiment, the stent-like structure may reduce the flow of lymph and chylomicron lipids through the thoracic duct completely (e.g., by 100 percent). The stent-like structure 10 may include a variety of different sizes such that the medical professional can select a pre-formed stent-like structure with the desired length 16a and/or diameter 16b of the medial portion 16 depending, for example, on the size of the patient and/or the amount of weight loss desired. The stent-like structure may include a variety of shapes and designs (e.g., laser-cut, braided etc.) and be formed from a variety of resilient insert materials, including metals and metal alloys such as platinum, tungsten, titanium, stainless steel, nickel and nickel-titanium alloys (e.g., nitinol), polymers such as acrylate-based polymers, polyurethane-based polymers, polynorbornene-based polymers, and polylactide-based polymers, and any combinations thereof. Other examples of polymers are disclosed, for example, in Buiser et al., U.S. Patent Pub. No. 20070141099, which is incorporated herein by reference in its entirety. In one embodiment, the stent-like structure may be expandable, using for example an inflatable balloon to move the stent-like structure from the first configuration to the second configuration. In another embodiment, the stent-like structure may be self-expandable such that the stent-like structure when released moves from the first configuration to the second configuration upon being deployed from a delivery catheter, such as by the withdrawal of a restraining sheath from about the outer diameter of the stent-like structure. In one embodiment, the stent-like structure may include a partial or complete coating (e.g., covering, sheath etc.) such that lymph and chylomicron lipids are unable to flow through the braided material that forms the body of the stent-like structure. Coating may also inhibit or reduce tissue in-growth that might otherwise hinder removal of the stent-like structure. In another embodiment, the stent-like structure may be biodegradable or bioabsorbable such that the flow of lymph and chylomicron lipids through the thoracic duct returns to the original (i.e., normal or unaltered) level after a pre-determined period of time.

The stent-like structure may be positioned within the thoracic duct using a variety of delivery systems and devices commonly known in the art. For example, a minimally invasive method may include a medical professional advancing a guidewire through the left subclavian vein into the thoracic duct. The stent-like structure may carried within a delivery catheter to maintain the stent-like structure in a first unexpanded configuration and advanced over the guidewire such that a distal end of the device is positioned at the desired location within the thoracic duct. The delivery catheter may then release the stent-like structure so that it moves from the first configuration (i.e., collapsed within the delivery catheter) to the second configuration within the thoracic duct. Once the stent-like structure is properly positioned within the patient, the delivery system and guidewire may be withdrawn from the patient.

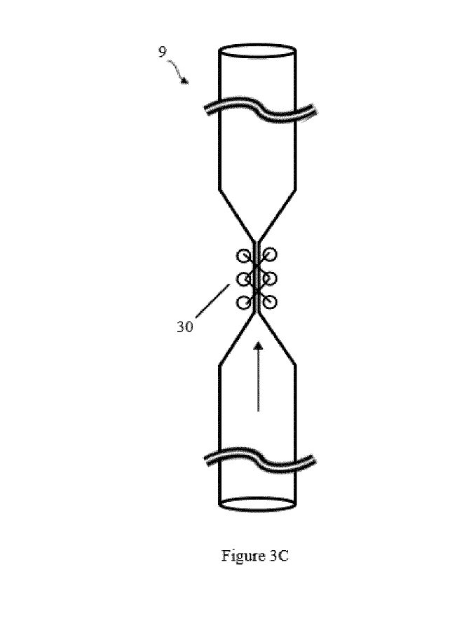

FIG. 3A generally depicts a stricture element 20 disposed circumferentially around an outer surface of the thoracic duct 9 distal to the intersection with the subclavian vein (not shown). The stricture element 20 may be placed at a variety of positions about the outer surface of the thoracic duct and/or the cysterna chyli. In one embodiment, the stricture element 20 may include a stricture or band configured to maintain a reduced profile of the duct and/or resist outward pressure from within the thoracic duct. Examples of such constrictive bands (e.g., lap-band, gastric bypass bands etc.) are known in the art, including, for example, U.S. Pat. No. 8,469,978; incorporated herein by reference in its entirety. The inner diameter of the constrictive band may range in size from approximately 1.0 mm to approximately 6.0 mm when in a relaxed (i.e., unstretched) configuration (e.g., from 2.0 mm to 5.0 mm; from 3.0 mm to 4.0 mm). In one embodiment, the constrictive band may be formed from a biodegradable or bioabsorbable material such that the flow of lymph and chylomicron lipids through the thoracic is temporarily reduced (or completely blocked), but returns to the original (i.e., normal or unaltered) level after a pre-determined period of time (e.g., once the desired weight loss is achieved).

Referring to FIG. 3B, in one embodiment the stricture element 20 may include a lumen 22 fluidly connected by a tube 26 to a fill-port 24 located, for example, underneath the patient's skin 28. The medical professional may adjust the amount of stricture of the thoracic duct 9 in an outpatient setting by flowing fluid from the fill-port 24 into the lumen 22 (i.e., to increase the amount of stricture) or by returning fluid from the lumen 22 to the fill-port 24 (i.e., to decrease the amount of stricture). FIG. 3C generally depicts another embodiment of a stricture element 30 disposed circumferentially around the outer surface of the thoracic duct. The stricture element includes a series of tie-offs (e.g., laces, sutures, strings, girdles, ratchets or related adjustable tensioning elements) that may be individually tensioned by the medical professional to achieve the desired amount of flow-reduction through the thoracic duct. For severely over-weight patients, this may include the complete closure of the thoracic duct to prevent any flow therethrough. As above, in one embodiment, the tie-offs may be formed from a biodegradable or bioabsorbable material such that the flow of lymph and chylomicron lipids through the thoracic is temporarily reduced (or completely blocked), but returns to the original (i.e., normal or unaltered) level after a pre-determined period of time (e.g., once the desired weight loss is achieved).

The stricture elements of FIGS. 3A-3C may be positioned around the thoracic duct using minimally invasive laparoscopic procedures as are known in the art. In addition, or alternatively, the stricture element(s) disclosed herein may be positioned via a minimally invasive natural orifice transluminal endoscopic surgery (NOTES) procedure. For example, the medical professional may advance an endoscope through an incision in the gastric lining such that the distal end of the endoscope is adjacent to the desired location of the thoracic duct. The stricture element may then be deployed through a working channel of the endoscope and circumferentially deployed around an outer portion of the thoracic duct, and the endoscope withdrawn from the patient. Compressive inward pressure or resistance may then be exerted on the thoracic duct via the stricture element as discussed above. As discussed above, the stricture element(s) of FIGS. 3A-3C may reduce the flow of lymph and chylomicron lipids through the thoracic duct by at least 10 percent; by at least 20 percent; by at least 30 percent; by at least 40; by at least 50 percent; by at least 60 percent; by at least 70 percent; by at least 80 percent; by at least 90 percent. In another embodiment, the stent-like structure may reduce the flow of lymph and chylomicron lipids through the thoracic duct completely (i.e., by 100 percent).

All of the devices and/or methods disclosed and claimed herein can be made and executed without undue experimentation in light of the present disclosure. While the devices and methods of this disclosure have been described in terms of exemplary embodiments, it may be apparent to those of skill in the art that variations can be applied to the devices and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

What is claimed is:

1. A method for affecting fluid flow through the thoracic duct of a patient, comprising:
contacting a stricture element with a portion of the thoracic duct to create a flow-affecting stricture therein, wherein the stricture element is placed within the thoracic duct.

2. The method of claim 1, wherein the stricture element reduces a flow of chylomicron lipids through the thoracic duct.

3. The method of claim 1, wherein the stricture element blocks a flow of chylomicron fluids through the thoracic duct.

4. The method of claim 1, wherein the stricture element is advanced to the thoracic duct through a vessel of the patient.

5. The method of claim 4, wherein the vessel includes a left subclavian vein.

6. The method of claim 1, wherein the stricture element includes:
a proximal end portion;
a distal end portion;
a medial portion; and
a lumen extending therebetween.

7. The method of claim 6, wherein the stricture element is configured to move from a first collapsed configuration to a second expanded configuration.

8. The method of claim 7, wherein a diameter of the lumen of the medial portion is less than a diameter of the lumen of the proximal and distal end portions when the stricture element is in the second expanded configuration.

9. A method for affecting fluid flow through the thoracic duct of a patient, comprising:
contacting a stricture element with a portion of the thoracic duct to create a flow-affecting stricture therein, wherein the stricture element is placed around the thoracic duct.

10. The method of claim 9, wherein the stricture element reduces a flow of chylomicron lipids through the thoracic duct.

11. The method of claim 9, wherein the stricture element blocks a flow of chylomicron fluids through the thoracic duct.

12. The method of claim 9, wherein the stricture element is placed around the thoracic duct laparoscopically.

13. The method of claim 9, wherein the stricture element includes a band configured to resist outward pressure on the thoracic duct creating a stricture.

14. The method of claim 13, wherein the band further includes a lumen fluidly connected to a fluid source.

15. The method of claim 14, wherein flowing a fluid from the fluid source into the lumen of the band increases a resistance to outward pressure or increases a constrictive pressure on the thoracic duct.

16. The method of claim 14, wherein flowing a fluid from the lumen of the band into the fluid source decreases a resistance to outward pressure or decreases a constrictive pressure on the thoracic duct.

17. A method for affecting fluid flow through a cysterna chyli of a patient, comprising:
contacting a stricture element with a portion of the cysterna chyli to create a flow-affecting stricture therein, wherein the stricture element is placed within or around the cysterna chyli.

18. The method of claim 17, wherein the stricture element reduces a flow of chylomicron lipids through the cysterna chyli.

19. The method of claim 17, wherein the stricture element blocks a flow of chylomicron fluids through the cysterna chyli.

20. The method of claim 18, wherein the stricture element reduces the flow of chylomicron lipids through the cysterna chyli by at least 50 percent.
